(19) United States
(12) Patent Application Publication
(10) Pub. No.: US 2004/0054400 A1
(43) Pub. Date: Mar. 18, 2004

(54) Conformable vascular stent

(76) Inventor: Vuan Fernando Granada, Pearland, TX (US)

Correspondence Address: Donald N. Halgren, 35 Central Street, Manchester, MA, 01944, US

(21) Appl. No.: 10/410,950

(22) Filed: Apr. 10, 2003

Related U.S. Application Data
(63) Continuation-in-part of application No. 09/624,812, filed on Jul. 25, 2000, now Pat. No. 6,679,910.
(60) Provisional application No. 60/165,279, filed on Nov. 12, 1999.

(51) Int. Cl. A61F 2/06

(52) U.S. Cl. 623/1.16

(57) ABSTRACT

A vascular conformable stent for implantation within a body lumen of a mammalian patient. The stent comprises a flexible tubular body having a first end and a second end. The tubular body is formed of a plurality of axially adjacent circumferential bands arranged axially therealong, a first of the bands being comprised of a zig-zag strand having a first edge and a second edge. The first edge is comprised of a cell end and a gap in an alternating sequence around the circumferential band, the second edge comprised of a gap and a cell end in an alternating sequence around the circumferential band. A second of the circumferential bands are comprised of a zig-zag strand having a first edge and a second edge, the first edge comprised of a cell end and a gap in an alternating sequence around the circumferential band, the second edge comprised of a gap and a cell end in an alternating sequence around the circumferential band. The second of the circumferential bands is axially adjacent to and is a mirror image of the first circumferential band.

### BACKGROUND OF THE INVENTION

1. Field of the Invention

This invention relates to intraluminal stents and more particularly to intravascular stents with improved conformability with the lumen in which it is placed, this application being a continuation-in-part application of my co-pending U.S. patent application Ser. No. 09/624,812, filed Jul. 25, 2000 which is based on provisional patent application serial No. 60/165,279 filed Nov. 12, 1999, each of which being incorporated by reference herein, in their entirety.

2. Prior Art

Stents are cylindrically shaped medical devices which are radially expandable for deployment by implantation into a body lumen or vessel for holding open a segment of that segment of that vessel or other anatomical lumen. Intraluminal stents have found a particular use in maintaining vessel patency following angioplasty, for example, in preventing restenosis of that stented vessel.

Intraluminal stents are typically inserted into a damaged vessel by the attending physician, by mounting the undeployed stent on a balloon catheter, and advancing the balloon catheter to the particular location within the patient's body, inflating the balloon on the catheter to expand the stent, and then deflating the balloon and removing the catheter from that vessel. The stent remains therein, in its deployed, expanded condition within the vessel and exerts a radial pressure on the vessel wall at the site of the lesion, to counter any tendency of the vessel to close.

In maintaining the vessel patency, a stent must also maintain conformity with the convolutions and bends within the vessel in which it has been placed. Unless a stent is able to bend in multiple directions, the stent itself may effect a closing of the vessel in which it has been placed.

It is an object of the present invention to provide a stent which overcomes the disadvantages of the prior art.

It is a further object of the present invention to provide a stent which is conformable to the vasculature in which it has been placed.

It is yet a still further object of the present invention to provide a stent which is able to bend in multiple directions, which stent has horizontal, vertical and torsional flexibility without the distortion of the stent, while still being able to provide a symmetric and controlled atraumatic expansion of that vessel in which it has been placed.

### BRIEF SUMMARY OF THE INVENTION

The present invention comprises a vascular conformable stent. The stent comprises a tubular body having a first end and a second end, the stent being formed by a plurality of circumferential bands including a first end band and a second end band and at least one intermediate band arranged therebetween. The circumferential bands are arranged axially, end to end. Each circumferential band is formed by a zig-zag or serpentine strand which are collectively manufactured preferably from a single elongated metal tube. Each of the opposing edges of each of these circumferential bands is formed by alternating “bends” or “cell ends”, and by “gaps”. In each circumferential band, a bend or cell end is opposed on its opposite edge by a gap. The cell ends on the edge of one of the plurality of circumferential bands are axially aligned with the cell ends forming the opposite edge of its adjacent circumferential band. Axially aligned bends or cell ends on adjacent circumferential bands are connected by “S” connectors or serpentine links. Each S connector or serpentine link comprises a pair of oppositely oriented curves. The serpentine links or S connectors between each pair of adjacent circumferential bands form a circumferential row. The rows of serpentine links or S connectors alternate between left handed rows and right handed rows. When the tubular body of the stent is viewed horizontally from left to right, the right handed rows comprise serpentine links or S connectors having a leftward most curve extending downwardly and a rightward most curve extending upwardly, and the left handed rows comprise serpentine links or S connectors having a leftward most curve extending upwardly and a rightward most curve extending downwardly.

A straight leg is arranged between each side of a cell end on a particular circumferential band on one edge thereof, and also on a side of an adjacent cell end on the other edge of that circumferential band. Each cell end is comprised of a generally circular strand of material defining an arc of about 270 degrees. The cell ends meet the straight leg members at a “pinched” portion, defined as a “cell end neck”. A pair of opposed cell ends and their respective straight leg members extending across a gap, merge into another cell end neck portion. Axially adjacent cell end neck portions are joined by the serpentine link or S connectors.

The stent of the present invention is radially expandable from a first cylindrical configuration of a first undeployed diameter to a radially enlarged cylindrical shape of a deployed second diameter. The stent is formed from a perforated tubular metal member to provide conformability in the body of the stent while providing rigidity in its forward leading and trailing edges thereof.

The multi-jointed arrangement of each unit cell being characterized by the serpentine links or S connectors minimizes any foreshorten-ing as the stent is being deployed by a balloon therewithin. The thickness of the material defining the serpentine link or S connector portions of the stent is thiner than the thickness of the material defining the respective cell ends or their adjacent straight legs of the stent. Each straight leg connects circumferentially adjacent cell ends on a particular circumferential band. Adjacent cell ends on a particular circumferential band are 180 degrees out of phase with one another. That is, their respective gaps alternate with respect to one another.

The legs of all the unit cells of all the circumferential bands comprise a general pattern of intersecting or crossed diagonally-arranged strands in both the deployed and the predeployed condition wherein those diagonal strands intersect at an acute angle with respect to the longitudinal axis of the stent itself.

Each band is defined by a cell end, a side of a neck portion, a straight leg to an adjacent neck portion, another cell end, another neck portion, and a straight leg and so on until the full circumference of the respective band is complete.

Another circumferential band is arranged adjacent the first band and is circumferentially out of phase so that one cell end of a first band is axially adjacent a cell end of the next band and is connected thereto by the serpentine link or S connector.

To enhance conformability of the stent when it is placed within a body vessel, each neck portion comprises a hinge section extendable and/or conformably adjustable in two directions. Each cell end comprises a further hinge section extendable and/or conformably adjustable in two directions. Each serpentine link or S connector, being thinner material than its adjacent cell end comprises a pair of hinged sections extendable and/or conformably adjustable in three directions, that is, bending the respective adjacent circumferential bands with respect to one another, providing axial expansion therewith, and providing tensional adjustability of each respective unit cell with any other unit cell of the stent.

Thus, each respective unit cell is defined by a pair of axially adjacent circumferential bands having opposed cell ends coming together at cell end necks, each cell end neck on its respective side of a cell end unitarily mating with a straight leg ending at a second cell end neck portion of a further cell end on the particular band. That cell end and neck arrangement being connected to a mirror image thereof by the serpentine length or S connector.

The unit cells, each being defined by a plurality of hinge portions and elements connected by thinner connectors permits a high range of expandability and conformable deployment from a thinner or narrow diameter undeployed state to a larger diameter fully deployed state in a convoluted vasculature.

The invention thus comprises a vascular conformable stent for implantation within a body lumen of a mammalian patient, comprising a flexible tubular body having a first end and a second end. The tubular body is formed of a plurality of axially adjacent circumferential bands arranged axially therealong. A first of the bands is comprised of a zig-zag strand having a first edge and a second edge, the first edge comprised of a cell end and a gap in an alternating sequence around the circumferential band, the second edge being comprised of a gap and a cell end in an alternating sequence around the circumferential band. A second of the circumferential bands is comprised of a zig-zag strand having a first edge and a second edge, the first edge being comprised of a cell end and a gap in an alternating sequence around the circumferential band, the second edge being comprised of a gap and a cell end in an alternating sequence around the circumferential band, wherein the second of the circumferential bands is axially adjacent to and is a mirror image of the first circumferential band.

Each of the circumferential bands are flexibly connected to an adjacent circumferential band by an “S” shaped connector. The “S” shaped connector may be radially thinner than the strands comprised of the zig-zag strands. The cell end comprises an arc of the strand extending through about 270 degrees. The gap comprises a circumferentially disposed open space between circumferentially adjacent cell ends. Each cell end has a pair of straight legs thereattached, at a pinched cell end neck thereon. A pair of axially aligned cell ends define a unit cell of the stent between them, each of the unit cells being of generally diamond shape having side edges generally defined by the straight legs extending from each of the cell ends. Each of the straight legs may be arranged at an acute angle with respect to the longitudinal axis of the stent. Each of the cell ends on the first circumferential band on the first end of the tubular body and each of the cell ends on the second edge of the circumferential band on the second end of the stent are free of the “S” shaped connector. The straight legs of the unit cells define a pattern of Xs in axial alignment along the outer surface of the tubular body of the stent, from the first end to the second end of the stent.

The invention may also comprise a method of deploying and conforming a vascular stent to any irregularities of a vascular lumen in a mammalian patient, comprising the steps of: arranging an array of unit cells along a foraminous tubular member, the unit cells being in axial alignment from a first end of the stent to a second end of the stent, the unit cells being attached in axial alignment to one another by an “S” shaped connector, the “S” shaped connector being radially thinner than the unit cells; deploying the vascular stent in a convoluted vessel; bending the vascular stent about the “S” shaped connector by deforming the “S” shaped connectors in a torsional direction, and in a radial direction while expanding the unit cells radially and circumferentially against the body lumen, to open the body lumen for facilitating the passage of a body fluid therethrough.

The method may include forming the unit cells by a pair of opposed cell ends in axial alignment with one another, and wherein each of the cell ends are in axial alignment with the longitudinal axis of the stent. Each of the cell ends may comprise a strand of material of arranged in a generally circular configuration extending through an arc of about 270 degrees. An annular array of unit cells is comprised of a pair of circumferential bands arranged in axial alignment with one another, each of the circumferential bands being connected to an axially adjacent circumferential band by the “S” connector between the unit cells. The unit cells deform in deployment by a twisting and elongation of the “S” shaped connectors and by a spreading open of the cell ends. The S connectors may be narrower and thinner than the strands of material comprising the cell ends.

### BRIEF DESCRIPTION OF THE DRAWINGS

The objects and advantages of the present invention will become more apparent when viewed in conjunction with the following drawings in which;

FIG. 1 is a side elevational view of a conformable vascular stent made according to the principles of the present invention, wherein the stent is shown separated along a longitudinal line at an arbitrary point laid out flat to better illustrate the preferred configuration;

FIG. 2 is a plan view of a unit cell extending across a pair of adjacent bands of a stent constructed according to the principles of the present invention;

FIG. 3 is a perspective view of a conformable vascular stent shown deployed in a non linear or curved orientation; and

FIG. 4 is an end view of an expanded conformable vascular stent constructed according to the principles of the present invention.

### DETAILED DESCRIPTION OF THE PREFERRED EMBODIMENTS

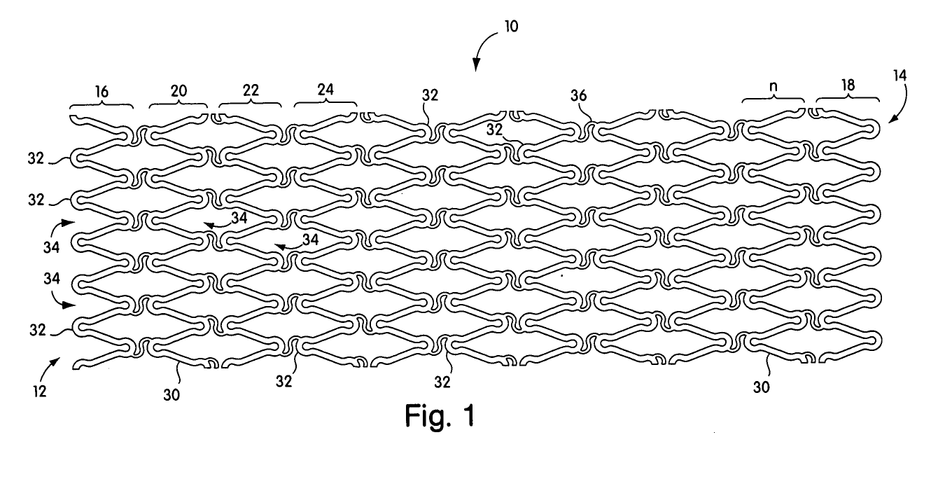

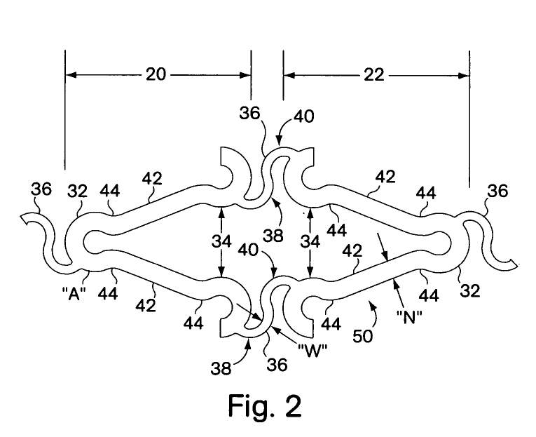

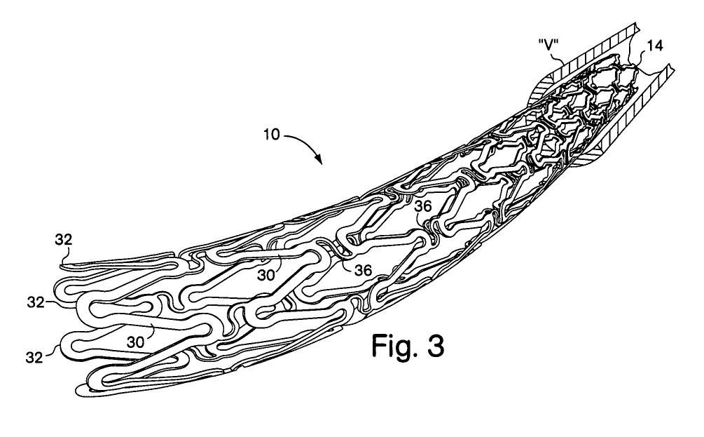

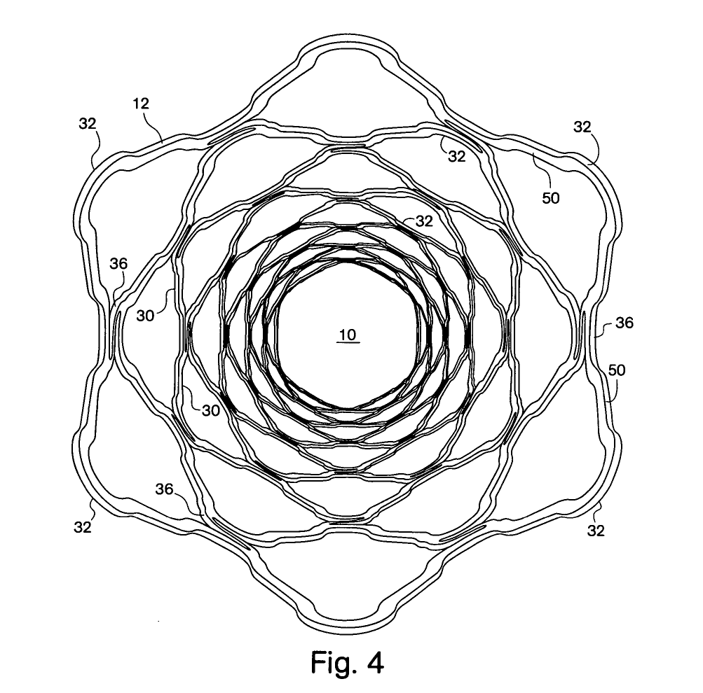

Referring now to the drawings in detail, and particularly to FIG. 1, there is shown the present invention in a “laid-out-flat” configuration, which invention comprises a unique vascular conformable stent 10. The stent 10 typically comprises a tubular body, which tubularity is shown more clearly however, in FIGS. 3 and 4. The stent 10 has a first end 12 and a second end 14, the stent 10 being formed by a plurality of circumferential bands including a first end circumferential band 16 and a second end circumferential band 18 and at least one intermediate circumferential band 20, 22, 24 . . . n arranged therebetween. The circumferential bands 16-24, n . . . are arranged axially, end to end, as represented in FIG. 1. Each circumferential band 16-n is formed by a zig-zag or serpentine strand 30 which are each collectively manufactured preferably from a single elongated metal tube, as may be more clearly seen in FIGS. 3 and 4. Each of the opposing edges of each of these circumferential bands is formed by alternating “bends” or “cell ends” 32, and by “gaps” 34. In each circumferential band 16 et seq., a bend or cell end 32 is opposed on its opposite edge by a gap 34, as is represented in FIGS. 1 and 2. The cell ends 32 on the edge of one of the plurality of circumferential bands 16-n are axially aligned with the cell ends 32 forming the opposite edge of its adjacent circumferential band. Axially aligned bends or cell ends 32 on adjacent circumferential bands 16-n are connected by “S” connectors or serpentine links 36. Each S connector or serpentine link 36 comprises a pair of oppositely oriented curves 38 and 40, as represented in FIG. 2. The serpentine links or S connectors 36 between each pair of adjacent circumferential bands 16-n form a circumferential row. The rows of serpentine links or S connectors 36 alternate between left handed rows and right handed rows, as may be seen in FIG. 1. When the tubular body of the stent 10 is viewed horizontally from left to right in FIG. 1, the right handed rows comprise serpentine links or S connectors 36 having a leftward most curve extending downwardly and a rightward most curve extending upwardly, and the left handed rows comprise serpentine links or S connectors 36 having a leftward most curve extending upwardly and a rightward most curve extending downwardly.

A straight leg 42, as shown in FIG. 2, is arranged between each side of a cell end 32 on each particular circumferential band 16-n on one edge thereof, and on a side of an adjacent cell end on the other edge of that circumferential band 16-n. Each cell end 32 is comprised of a generally circular strand of material 30 defining an arc “A” of about 270 degrees. The cell ends 32 meet their respective straight leg members 42 at a “pinched” portion, defined as a “cell end neck” 44, as best represented in FIG. 2. A pair of opposed cell ends 32 and their respective straight leg members 42 extending across a gap 34, then each merge into another cell end neck portion 44. Axially adjacent cell end neck portions 44 are joined by the serpentine link or S connectors 36.

The stent 10 of the present invention is radially expandable from a first cylindrical configuration of a first undeployed diameter to a radially enlarged cylindrical shape of a deployed second diameter, as represented in FIGS. 3 and 4. The stent 10 is preferably formed from a perforated or machined tubular metal member to provide conformability in the body of the stent 10 while providing rigidity in its forward leading and trailing edges 12 and 14 thereof.

The multi-jointed arrangement of each unit cell 50 being characterized by the serpentine links or S connectors 36 minimizes any foreshortening as the stent 10 is being deployed by a balloon therewithin. In one preferred embodiment, the thickness of the material defining the serpentine link or S connector portions 36 of the stent 10 is thinner than the thickness of the strand of material 30 defining the respective cell ends 32 or their adjacent straight legs 42 of the stent 10. In another preferred embodiment, the width “W” of the S connector portions 36 is about 50% of the width “N” of the strand of material 30, as represented in FIG. 2. Each straight leg 42 connects circumferentially adjacent cell ends 32 on a particular circumferential band 16-n. Adjacent cell ends 32 on a particular circumferential band 16-n are 180 degrees out of phase with one another. That is, their respective gaps 34 and cell ends 32 alternate with respect to one another.

The legs 42 of all the unit cells 50 of all the circumferential bands 16n comprise a general “XXX” pattern of intersecting or crossed, diagonally-arranged strands 30 in both the deployed and the pre-deployed condition wherein those diagonal strands (pattern of legs 42) intersect at an acute angle with respect to the longitudinal axis “L” of the stent 10 itself.

Each band 16-n is thus defined by a cell end 32, a side of a neck portion 44, a straight leg 42 to an adjacent neck portion 44, another cell end 32, another neck portion 44, and a straight leg 42 and so on until the full circumference of the respective band 16-n is complete.

Another circumferential band 20 is arranged adjacent the first band 16 and is circumferentially out of phase so that one cell end 32 of the first band 16 is axially adjacent a cell end 32 of the next band 20 and is connected thereto by the serpentine link or S connector 36 therebetween.

To enhance conformability of the stent 10 when it is placed within a body vessel “V”, each neck portion 44 comprises a hinge section extendable and/or conformably adjustable in two directions (longitudinally and circumferentially). Each cell end 32 comprises a further “hinge section” extendable and/or conformably adjustable in two directions (circumferentially and radially). Each serpentine link or S connector 36, being manufactured of thinner and/or narrower material than its adjacent cell end 36 comprises a pair of hinged sections extendable and/or conformably adjustable in three directions (circumferentially/torsionally, longitudinally and radially), that is, permitting the bending and twisting of the respective adjacent circumferential bands 16-n with respect to one another, permitting axial and radial expansion/contraction therewith, and permitting/providing torsional adjustability of each respective unit cell 50 with any other unit cell 50 of the stent 10.

Thus, each respective unit cell 50 is defined by a pair of axially adjacent circumferential bands 16-n having opposed cell ends 32 coming together at cell end necks 44, each cell end neck 44 on its respective side of a cell end 32 unitarily mating with a straight leg 42 ending at a second cell end neck portion 44 of a further cell end 32 on the particular band 16-n. That cell end 32 and neck arrangement 44 being connected to a mirror image thereof by the serpentine length or S connector 36. The unit cells 50, each being defined by a plurality of hinge portions and elements connected by thinner material connectors 36 permits a high range of expandability and a conformable deployment from a thinner or narrow diameter undeployed state to a larger diameter fully deployed state in a convoluted vasculature. The cell ends 32 on each end 12 and 14 of the stent 10 also provide stent conformability by their ability to expand and conform to the desired deployed state without directly adjacent circumferential attachment to a neighboring cell end. From the first end cell, longitudinally to the second end cell to the gap in the first band to the gap to the cell in the second band to the S shaped connector to the cell to the gap in the third band to the next gap to the next cell in the fourth band to the next S shaped connector etc defines the longitudinal characteristics of the stent of the present invention.

1. A vascular conformable stent for implantation within a body lumen of a mammalian patient, comprising:
a flexible tubular body having a first end and a second end, said tubular body formed of a plurality of axially adjacent circumferential bands arranged axially therealong, a first of said bands comprised of a zig-zag strand having a first edge and a second edge, said first edge comprised of a cell end and a gap in an alternating sequence around said circumferential band, said second edge comprised of a gap and a cell end in an alternating sequence around said circumferential band;
a second of said circumferential bands comprised of a zigzag strand having a first edge and a second edge, said first edge comprised of a cell end and a gap in an alternating sequence around said circumferential band, said second edge comprised of a gap and a cell end in an alternating sequence around said circumferential band;
wherein said second of said circumferential bands is axially adjacent to and is a mirror image of said first circumferential band.

2. The vascular conformable stent as recited in claim 1, wherein each of said circumferential bands are flexibly connected to an adjacent circumferential band by an “S” shaped connector.

3. The vascular conformable stent as recited in claim 2, wherein said “S” shaped connector is radially thinner than said strands comprised of said zig-zag strands.

4. The vascular conformable stent as recited in claim 2, wherein said “S” shaped connector is narrower than said strands comprised of said zig-zag strands.

5. The vascular conformable stent as recited in claim 3, wherein said “S” shaped connector is at least about 50% narrower than said strands comprised of said zig-zag strands

6. The vascular conformable stent as recited in claim 1, wherein said cell end comprises an arc of said strand extending through about 270 degrees.

7. The vascular conformable stent as recited in claim 1, wherein said gap comprises a circumferentially disposed open space between circumferentially adjacent cell ends.

8. The vascular conformable stent as recited in claim 6, wherein said cell end has a pair of straight legs thereattached, at a pinched cell end neck thereon.

9. The vascular conformable stent as recited in claim 8, wherein a pair of axially aligned cell ends define a unit cell of said stent between them, each of said unit cells being of generally diamond shape having side edges generally defined by said straight legs extending from each of said cell end.

10. The vascular conformable stent as recited in claim 9, wherein each of said straight legs are arranged at an acute angle with respect to the longitudinal axis of said stent.

11. The vascular conformable stent as recited in claim 1, wherein each of said cell ends on said first circumferential band on said first end of said tubular body and each of said cell ends on said second edge of said circumferential band on said second end of said stent are free of said “S” shaped connector.

12. The vascular conformable stent as recited in claim 10, wherein said straight legs of said unit cells define a pattern of Xs in axial alignment along the outer surface of said tubular body of said stent, from said first end to said second end.

13. A method of deploying and conforming a vascular stent to any irregularities of a vascular lumen in a mammalian patient, comprising the steps of:
arranging an array of unit cells along a foraminous tubular member, said unit cells being in axial alignment from a first end of said stent to a second end of said stent, said unit cells being attached in axial alignment to one another by an “S” shaped connector, said “S” shaped connector being radially thinner than said unit cells;
deploying said vascular stent in a convoluted vessel;
bending said vascular stent about said “S” shaped connector by deforming said “S” shaped connectors in a torsional direction, and in a radial direction while expanding said unit cells radially and circumferentially against said body lumen, to open said body lumen for facilitating the passage of a body fluid therethrough.

14. The method as recited in claim 13, including:
forming said unit cells by a pair of opposed cell ends in axial alignment with one another, and wherein each of said cell ends are in axial alignment with the longitudinal axis of said stent.

15. The method as recited in claim 14, wherein each of said cell end comprises a strand of material of arranged in a generally circular configuration extending through an arc of about 270 degrees.

16. The method as recited in claim 15, wherein an annular array of unit cells is comprised of a pair of circumferential bands arranged in axial alignment with one another, each said circumferential bands being connected to an axially adjacent circumferential band by said “S” connector between said unit cells.

17. The method as recited in claim 16, wherein said unit cells deform in deployment by a twisting and elongation of said “S” shaped connectors and by a spreading open of said cell ends.

18. The method as recited in claim 17, wherein said S shaped connectors are radially thinner and about 50% narrower than said strands of material comprising said end cells to facilitate longitudinal, torsional and rotational adjustment and conformance of said vascular stent.
